(19) United States
(12) Patent Application Publication
(10) Pub. No.: US 2022/0096375 A1
(43) Pub. Date: Mar. 31, 2022

(54) STABILIZED NON-ENVELOPED VIRUS COMPOSITIONS

(71) Applicant: ZICCUM AB, Huddinge (SE)

(72) Inventors: Göran Conradson, Bjärred (SE); Fernando Acevedo Fonseca, Märsta (SE)

(21) Appl. No.: 17/421,116

(22) PCT Filed: Jan. 9, 2020
(86) PCT No.: PCT/SE2020/050011

§ 371 Date: Jul. 7, 2021

(30) Foreign Application Priority Data
Jan. 9, 2019 (SE) 1950019-8

(51) Int. Cl. A61K 9/00; A61K 9/16; A61K 39/235

(52) U.S. Cl. CPC A61K 9/0073; A61K 2039/5252; A61K 39/235; A61K 9/1652

(57) ABSTRACT

The present invention relates to methods and compositions of thermostable nonenveloped virus as obtained by a laminar counter-current spray drying process. The compositions comprise aerosolizable amorphous particles, comprising free, non-encapsulated nonenveloped virus and an excipient, wherein the particles typically have a mass median aerodynamic diameter (MMAD) of less 5 μm and comprise less than 5% water.

### TECHNICAL FIELD

The present invention generally relates to a thermostable aerosolizable dry non-enveloped virus composition comprising nonenveloped virus produced by a laminar flow spray drying process.

### BACKGROUND OF THE INVENTION

Vaccines are generally distributed from manufacturers to users under carefully controlled conditions recommended by WHO guidelines that includes a cold chain. Both for vaccines and other biological pharmaceuticals stability during storage and distribution is a common problem especially when there is a vaccination requirement in remote areas with limited or no access to a cold chain. For this reason, dry powder vaccines (DPVs) are attractive, as they potentially may be more storage stable, but generate considerations to the producing drying processes and subsequent packaging and delivery systems. The article by T. F. Bahamondez-Canas et al. in Eur. J. Pharm and Biopharm., 2018, Vol. 122, pp 167-175 outlines the need for stable vaccines that are administrable intranasally or pulmonary, while reviewing contemporary efforts in providing such systems. It also discussed in G Kanojla et al; Human vaccines & Immunotherapeutics, 2017, Vol. 13(10), pp 2364-2378 how spray drying of vaccines may be alternative to freeze-drying to provide an administrable powders. However, drawbacks with stress factors that affect the activity of the biomaterial are also outlined and it is obvious that there is a need for processes that both better retain the viral activity and lead to high quality aerosolizable powder formulations in order to establish vaccine that can be conveniently administered in different administration routes with effective dose control. WO 2017/035664 discloses a stabilized adenovirus composition comprising dextran and mannitol that is reported to retain at least 40% adenovirus activity at ambient temperature storage. However a rather low yield is reported and it is not disclosed if a high quality inhalable aerosol can be formed without the tendency of re-agglomeration. U.S. Pat. No. 9,839,613 disclose dry formulation of a vaccine with a live attenuated virus, a non-reducing sugar, an amino acid stabilizer and a protein stabilizer. It is not reported if the composition is suitable to form an aerosol and the maintained activity appears to be rather low. U.S. Pat. No. 8,347,525 discloses a spray drying apparatus and a process for producing micronized powders of liable pharmaceuticals. However, it is not disclosed how to formulate vaccines or biological products that both are aerosolizable and thermostable while retaining effective activity following the drying process and subsequent storage in ambient temperatures. US2018161415 discloses thermostable dry adenovirus compositions relying on excipients comprising mannitol and dextran, as protectors during conventional spray drying methods. It is however, obvious that losses in viral activity is nevertheless obtained with these methodologies and that there is a need for new methods of and compositions that improve stability in dry form virus compositions, also throughout prolonged storage at ambient temperatures.

Viruses are generally divided in nonenveloped and enveloped types. Both types of virus particles contain the viral genome packaged in a protein coat called the capsid. Enveloped viruses are surrounded by an outer lipid bilayer membrane frequently comprising, glycoproteins, whereas nonenveloped viruses lack this membrane. The capsid and the envelope are instrumental in the various mechanisms of viral infection of cells and the transfer of viral genetic material between cells. The fragility of the envelope means that such types of virus are considered to be more sensitive to changes in temperature and other environmental factors. However, the structures of the virus surfaces of non-enveloped virus, such as adenovirus are also very sensitive to conformation changes and interaction with different agents used to formulate virus or viral parts. It would therefore be desirable to provide methods and formulations that counteract loss or reduction of desirable activity during preparation and storage of virus products, e.g. vaccines.

### SUMMARY OF INVENTION

It is a general object of the present invention is to provide a dry nonenveloped virus composition comprising a nonenveloped virus and an excipient that is thermostable and can be stored at temperatures of at least 40° C. and maintain an effective activity.

It is an object of the present invention to provide a dry nonenveloped virus composition that is aerosolizable and thereby suitable as an inhalable drug, for example by pulmonary deposition, or suitable for intranasal administration by effect of the qualities of the dry particulate composition.

It is also an object of the present invention to provide a dry composition that comprise free non-encapsulated nonenveloped produced without such agents that contribute to deposit on the virus surface, encapsulate the virus or aggregate the virus and thereby contribute to inactivation during the drying process and/or the subsequent storage.

It is also an object of the invention to provide processes and resulting dry nonenveloped virus compositions that obtains thermostability at 40° C. independent of how the virus originally has been manufactured and formulated with excipients.

It is one important purpose of the invention that the nonenveloped virus of the dry compositions shall retain sufficient activity to elicit an immune response or a therapeutic response and thereby be capable of acting as a vaccine or a pharmaceutical. The skilled person will be able to construe numerous applicable systems with a wide variety of antigens including but not limited to those associated with infectious diseases derived from various viruses, bacteria and fungi etc. such as those listed, for example in EP 2432502.

The nonenveloped virus can be a live nonenveloped virus, a live attenuated nonenveloped virus, an inactivated nonenveloped virus, a fragment or a subunit of a nonenveloped virus and may comprise a recombinant nonenveloped virus employed as a vector encoding at least one protein with antigen or therapeutic characteristics. The nonenveloped virus of the present invention originate from the groups of dsDNA, ssDNA, ssRNA+, ssRNA−; ssRNA=>DNA, and dsDNAs=>ssRNA.

The dsDNA nonenveloped virus, suitably is an adenovirus, such as AdV5 or EDS or a Papillomaviridae, such as HPV. For a ssDNA noneveloped virus, it is suitably a Parovirus. For a dsRNA nonenveloped virus, it is suitably a reovirus, a orthoreovirus or a rotavirus. For a ssRNA+ nonenveloped virus, it is suitably a Picorniaviridae, an enterovirus such as EV68, poliovirus, FMD virus or rhinovirus, or a Matonaviridiae, such as Rubella virus. For a ssRNA− nonenveloped virus, it is suitably a Rhabdovirus. For a ssRNA=>DNA nonenveloped virus, it is suitably a retrovirus. Generally, in the context of the invention the term nonenveloped virus refers to the virus itself and all derivatives or modifications thereof including serotypes, subtypes in natural or recombinant form. Preferably, the nonenveloped virus of the inventive compositions is an adenovirus.

The compositions comprise an excipient, which in the context of the present invention generally refers to all agents used to provide a form or shape of the inventive composition, but does not refer to the virus particles. The term excipient will have the same meaning as a pharmaceutically acceptable carrier, and it is of importance for the inventive compositions and processes that the excipient can act as a bulking agent throughout the drying process and in the subsequently obtained dry composition comprising virus particles. The excipient may thereby to contribute to stabilize the virus in the drying process and in the dry composition for storage. The excipient is selected so that encapsulation of the virus is avoided, which for example means that such inclusive lipid particles or crystals shall not be formed that may negatively interact with liable surface structures of the virus. For the same reason, the excipient preferably is selected so it does not promote aggregation of virus units and not form any deposits on the virus surface. Accordingly, virus as included in the inventive dry composition comprise essentially free non-aggregated virus units without any surface deposition, distributed and dispersed in composition particles.

In a first general aspect, the invention is directed to a thermostable dry nonenveloped virus composition comprising aerosolizable amorphous particles, comprising essentially free, non-encapsulated nonenveloped virus and an excipient, wherein the particles have a mass median aerodynamic diameter (MMAD) of less than 5 μm, preferably from about 1 to 5 μm, such as 2 to 3 μm; while the composition comprises less than 5% (wt) of water.

In this context of the invention, the term “aerosolizable” has the meaning that the compositions shall be applicable for use to form a dry aerosol composition with suitable characteristics, such as mass median aerodynamic diameter (MMAD), that is suitably homogenous and coherent following administration with state of the art metered dose inhalers (MDIs) to obtain an effective pulmonary deposition, i.e. to admit the provision of an inhalable vaccine.

Preferably, the enveloped virus in the dry particles maintain activity or pharmacologically effective activity of their activity following storage at 40° C. for at least seven weeks. The activity suitably is vaccine activity or pharmacologic activity.

In the general context of the invention, the term “activity” means that the virus may be considered as antigenicity. Preferably, such maintained antigenicity that is demonstrable with a Hemagglutination assay (HA), as outlined in the experimental part below.

In some embodiments, the dry compositions are further adapted as inhalable compositions, for example for pulmonary administration and comprise suitable conventional pharmaceutical agents for this purpose, including surfactants, preservatives and additional stability increasing agents.

According to general aspects of the invention, the excipients of the composition comprises at least one cyclodextrin. The weight dry composition of the invention will predominantly arrive from the excipient and typically the dry compositions will include 70 to 100% (wt) of a cyclodextrin, preferably at least 90% (wt) of cyclodextrin, and more preferably at least about 95% (wt).

Suitable cyclodextrins for the invented compositions can be found in the document https://www.ema.europa.eu/documents/report/background-review-cyclodextrins-used-excipients-context-revision-guideline-excipients-label-package_en.pdf.

The cyclodextrins of the excipient are selected so that their three-dimensional structures and differences in hydrophobicity and hydrophilicity contribute to an arrangement of protecting the virus particles developed during the drying process, as further described, below and throughout subsequent storage of the dry compositions. Preferably, the cyclodextrin is 2-hydroxypropyl-beta-cyclodextrin (HP-β-CD). However, other similarly derivatized cyclodextrins with a comparably favourable three-dimensional structure and distribution of hydrophobicity and hydrophilicity would be conceivable with the invention.

In suitable dry compositions according to the invention, the added excipient comprises substantially only cyclodextrin and residues from a buffering agent. However, the excipient may comprise other conventional agents used in small amount such less than 30%, less than 20%, less than 10%, less than 5% or less than 1% (all wt.), preferably present so small amounts and/or selected so that such an agent does not interfere with establishment of the protective arrangement of cyclodextrin molecules in combination with the virus particles during the drying process.

In certain embodiments of the invention, the excipient of the composition is free from amino acids, such as those commonly used for example in lyophilisation of biomaterials, such as leucine and glycine. In the present context of providing a dry thermostable aerosolizable composition of enveloped virus, it appears that certain amino acids may compromise the quality of the dry particles and generate production problems, for example when collecting the dry particles.

In certain embodiments of the invention, the excipient is free from monosaccharides, oligosaccharides, higher saccharides, polysaccharides (such as dextran) and sugar alcohols, especially such compounds with a capacity to crystallize or risk to encapsulate the virus and thereby compromise the virus surface structure. Preferably, according to these aspects, the excipient is free from glycine and mannitol.

In this context, excipient free from amino acids and saccharides means that the inventive compositions are free from such added excipients, but that minor amounts can be present from buffers or from an original virus formulation constituents.

In one aspect, the composition is made with a spray drying process comprising providing an aqueous nonenveloped virus composition comprising 0.5 to 5% (wt) of at least one cyclodextrin and nebulizing it into droplets and transporting it in a laminar gas flow separated from a drying gas in a laminar opposite flow (opposite flow direction), while admitting vapour to diffuse through a membrane separating said gas flows, and finally collecting the dry powder composition.

Accordingly, methods of preparing a thermostable, aerosolizable dry nonenveloped virus composition by a counter-current spray drying process can comprise the steps of:

providing a liquid composition comprising nonenveloped virus and an excipient, nebulizing said composition into transportable droplets of less than 50 μm in a tube reactor having an inner region and an outer region;
admitting the nebulized composition to descend in a laminar carrier gas flow, while admitting a laminar flow of dry gas to ascend in said outer region in order to establish a counter-current drying of the descending drops; and
drying the droplets during a time period of 30 seconds to 2 minutes at an ambient temperature from 15 to 30° C., while admitting vapour to diffuse into the ascending laminar flow of dry air, and collecting a dry composition of particles as previously disclosed.

In one aspect of the method the inner region and the outer region tube reactor are separated by perforated process tube with an outer periphery covered by a membrane configured to admit vapour to diffuse through the membrane into the ascending laminar flow of dry air at a rate that exceeds an opposite flow rate of dry gas radially through the membrane.

Preferably the diffusion rate is higher than the opposite radial flow rate of dry gas through the membrane.

The laminar counter-current spray drying as previously outlined with the present invention is suitably performed with an equipment and methods outlined to in U.S. Pat. No. 8,347,525 and in S Soltani et al in Chemical Engineering Research and Design 0263-8762 (ISSN) 1744-3563 (eISSN) Vol. 93 p. 163-173.

In some embodiments, the liquid composition of nonenveloped virus comprises 0.5 to 5% (wt) of cyclodextrin, preferably 2-hydroxypropyl-beta-cyclodextrin.

In some embodiments, the ascending flow rate of drying gas is higher than the descending flow rate.

In some embodiments the ascending flow swirls or rotates around the membrane.

In some embodiments, the liquid virus composition has been subjected to a pre-treatment step. Suitably, such excipients are removed that may contribute to at least one of viral encapsulation, viral aggregation, crystallization and such aggregation of the resulting composition particles that counteracts aerosolization. Examples of such excipients are detergents, lipids, phospholipids, certain sugars and other debris from cells and other hydrophobic agents that risk to negatively counteract with the nonenveloped virus surfaces.

In some embodiments of the inventive methods, the liquid virus composition has been subjected to treatment with a surfactant and removal of micelles with such undesired hydrophobic agents. Suitably such a surfactant can be a cholate, such as sodium cholate.

The pre-treatment step or steps may enable the usefulness of the invention to provide thermostable dry compositions also from such commercial virus products that are formulated as liquids for cold storage or other normally temperature liable nonenveloped virus products.

Finally, the invention is directed to any of the previously disclosed dry compositions produced by any of the disclosed methods.

### BRIEF DESCRIPTION OF DRAWINGS

The invention is now described, by way of example, with reference to the accompanying drawings, in which:

FIGS. 1 to 3 show Hemagglutinin assays of viruses in compositions according to the invention.

FIG. 4 shows particle size distribution in a composition according to the invention.

FIG. 5 shows a scanning electron micrograph of virus particles in stored compositions formulated and prepared according to the invention.

FIG. 6 shows an example of a hemagglutination assay used with Example 2.

### DETAILED AND EXEMPLIFYING DESCRIPTION OF THE INVENTION

### Example 1

### Materials and Methods.

Inactivated EDS Adenovirus: From Hester Biosciences Limited, Gujarat, India (www.hester.in) and from the State Veterinary Office of Sweden, Uppsala (SVA).

The iEDS virus preparation from SVA was provided as a clear suspension of virus with a Hemagglutinin Assay (HA) titer of 64-128.

The Virus preparation from Hester was formulated as a vaccine. The ideas virus from the vaccine was extracted with a 5% solution of Sodium Cholate in a double extraction procedure. The cholate in the clear second extract was removed by a chromatographic separation on Sephadex G-75 equilibrated with 7 mM Phosphate buffer pH 7.5. The material eluting in the void volume of the gel was assessed by HA analysis of the virus activity and the active fractions were pooled.

A 20% solution of 2-hydoxypropyl-beta-Cyclodextrin was added to either virus suspension to a final concentration of 2%. A sample of 5 mL of the suspension was then applied to the nebulizer of the Laminar Pace instrument and spray dried (see http://www.inhalation.se/produkter/laminarpace/). The settings were: Nebulizer pulse rate 5%, nebulize air flow of 2.5 L/min, counterflow drying air 4.2 L/min. The drying process lasted for about 2-3 hours. The dried material was collected on a pre-weighted nylon filter at the bottom of the Laminar Pace column. At the end of the drying process the filter was weighed. The amount of dried material corresponded to 5 mL sample for later dilutions for analysis. The total yield was in most cases over 80% of material. Small samples of the dried powder were aliquoted in glass vials and sealed for analysis and stability studies at −20° C., 4 C, RT and 40° C.

The specificity of the HA analysis was controlled by hemagglutinin inhibition assay (HIA) using sera of iEDS immunized chicken as inhibitor.

To measure the aerodynamic particle size distribution of the spray dried material the powder was aerosolized and passed on a 9-step cascade impactor at an air flow of 2 L/min.

### Results

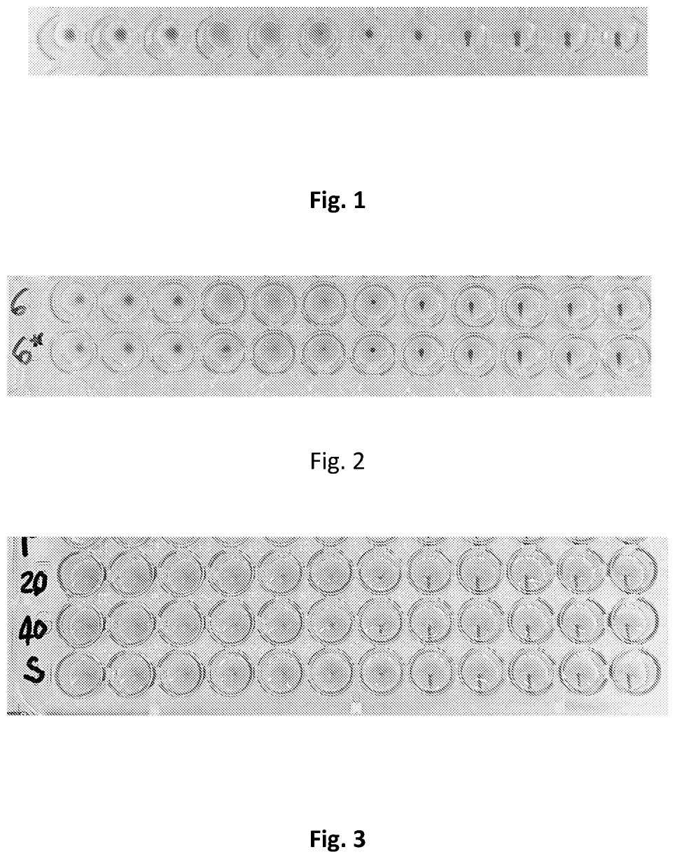

Chicken erythrocyte hemagglutinin (HA) assay of a reconstituted sample of vaccine extract derived from the EDS adenovirus obtained from Hester after Sephadex G-75 separation, spray dried in 2-hydroxypropyl-beta-cyclodextrin. The titer 128 is shown in FIG. 1

An additional step in lipidic excipient material removal from the virus suspension sample, as above, by treatment with paraffin oil, slightly improved the test result to a titer 128-256, see FIG. 2 for the HA analysis of samples as above kept at 4 C (6) and 40 C (6*) for one week.

Chicken erythrocyte hemagglutinin assay of a reconstituted samples of Inactivated EDS virus provided by the State Veterinary Office of Sweden, Uppsala, spray dried in 2-hydroxypropyl-beta-cyclodextrin stored one week at RT (20); one week at 40 C (40) compared to the original non dried material kept at 4 C. The result of the HA-test is shown in FIG. 3.

The results demonstrated in FIGS. 1-3 indicate no significant hemagglutinin activity loss by the spray drying process at 40° C. of dry compositions from both sources.

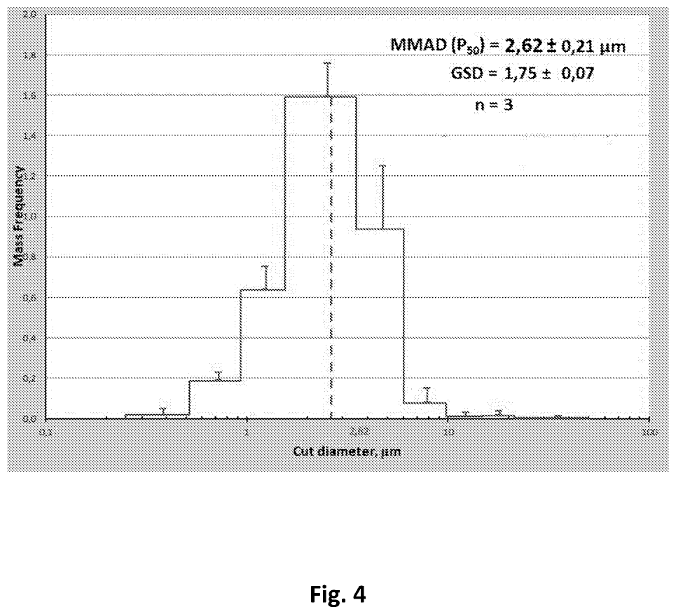

The aerodynamic particle size distribution in triplicate of a sample of spray dried inactivated EDS virus (from SVA) is illustrated in FIG. 4. The result indicates that the inactivated EDS virus powder can generate respirable aerosols with a Mass Median Aerodynamic Diameter (MMAD) of 2.62 microns.

### Study of Egg Drop Syndrome Virus Particles According to the Invention by Scanning Electron Microscopy and Negative Staining/Transmission Electron Microscopy

A dry composition of the iEDS virus preparation from SVA according was prepared with the methods as previously described.

Samples of Egg Drop Syndrome Virus particles were subjected to negative staining with 2% uranyl acetate on carbon-coated grids that were rendered hydrophilic by glow discharge at low pressure in air. Specimens were examined in a Philips/FEI CM 100 BioTWIN transmission electron microscope at the FCIM, Panum Institute, Copenhagen University. Images were recorded with a side-mounted Olympus Veleta camera with a resolution of 2048×2048 pixels (2 k×2K). In parallel, samples were prepared for scanning electron microscopy by sputtering with 30 nm gold/palladium in a Leica Coater ACE200 and visualized in a FEI Quanta 3D FEG scanning electron microscope at the FCIM, Panum Institute, Copenhagen University.

The resulting scanning electron micrographs exhibit narrow range size distributions of droplet structures up to 1 μm in diameter, present in all virus samples at 5.000× magnification and 15.000× magnification.

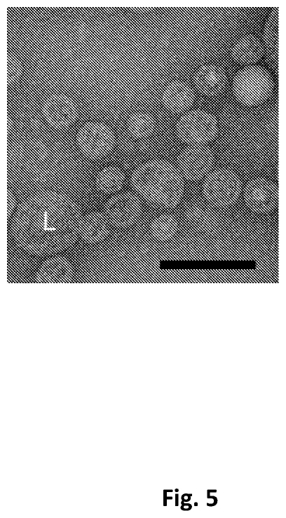

Negative staining and transmission electron microscopy show a uniform background of virus particles in all samples, about 80 nm in size, often exhibiting a hexagonal-like shape. FIG. 5 shows a sample of a composition that has been stored at 40° C. for one week. The virus particles are about 80 nm in diameter and a larger membrane structure resembling lipids is observed in all samples (L). The scale bar represents 200 nm. FIG. 5 demonstrates that the composition and methods according to the invention are capable of generating free non-encapsulated virus without any deposits on the virus surfaces. The virus also appear to retain its original morphology also after storage at an elevated temperature and following the process of proving a dry composition. From the fact that apparent lipid structures remain obviously from the original formulation of EDV, it is concluded that the invention is capable of providing free sufficiently viable and dry adenovirus compositions irrespectively of the original source or formulation.

### Example 2

Example 2 compares EDS virus activity before and after a spraying drying method and excipients according to the invention. In Example 2, inactivated EDS virus was acquired from GD animal health and the following protocol was use for the haemagglutination (HA) tests for virus activity.

### Hemagglutination Assay (HA)

### Equipment and Materials

The following equipment was used. Tabletop centrifuge with appropriate fittings. Red blood cells from appropriate host in Alsevers solution (Sigma-aldrich code A3551). Alsevers solution is composed of 4.2 g/L NaCl, 8.0 g/L citric acid.3Na.2H₂O, 0.55 g/L Citric acid.H2O, 20.5 g/L D-glucose, and used as anticoagulant/blood preservative. V-shaped bottomed 96-well plates, PBS solution

### Red Blood Cell Preparation

Chicken blood was bought at Håtunalab AB (https://www.hatunalab.com/en-GB). For chicken blood order 2 ml of whole blood, this should be mixed with 2 ml of Alsevers solution.

### Preparation of RBC Solution

1. Spin down the 4 ml; 2. Add 9 ml ice-cold 1×PBS, mix by inverting, spin down; 3. Add 9 ml ice-cold 1×PBS, mix by inverting, spin down; 4. Add 9 ml ice-cold Alsever, mix by inverting, spin down. Spin down at 1000 rpm for 10 minutes, corresponding to approximately 0.2 rcf or 200 g. Remove supernatant after step 1, 2, 3 and 4.

### Measure Hematocrit

This measurement is done after the third wash with Alsevers solution. The packed cell volume of the red blood cells from the whole blood is estimated by having an identical tube, where water is added, until the packed cell volume and the water level is at the same level. In general—the packed cell volume from 2 ml of whole blood is between 750 to 800 μl, which would correspond to a packed cell volume/hematocrit of minimum 37.5%, estimated from (750 μl/2000 μl)*100%. Based on the packed cell volume, the amount of Alsevers solution to be added, can be estimated in order to make a 10% RBC solution.

### Preparing a 1% RBC Solution for HA Assay

In general, a 1% (v/v) solution of RBC is used for the HA assay.
1. Take 1 ml of 10% RBC in Alsevers solution.
2. Add 9 ml of 1×PBS/alternative buffer.
3. This solution is ready to be used for HA assay.

### Viral Dilution and Assay

1. A V-shaped bottomed 96-well microtiter plate is preferred for this assay.
2. To each well, add 25 μl 1×PBS/alternative buffer with 1 mM Tris and 75 nM NaCl.
3. In the first column, add 25 μl of virus/vaccine sample.
4. Mix each well and transfer 25 μl to the next well on its right. Repeat mixing and transferring 25 μl down the length of the plate until well 11. Discard 25 μl from this well. Well 12 consist of pure PBS/buffer and serves as a negative control.
5. Add 25 μl of 1% red blood cells working solution to each well. Mix gently
6. Leave at room temperature for 30-60 minutes to develop. Negative results will appear as dots also called buttons (pellet of red blood cells) in the center of the plate. Positive results will form a uniform reddish color across the well.
7. Place the plate in an upright position in order to distinguish between dot and running dot.
8. Both dots and running dots should be reported.
9. Interpretation—the virus/vaccines titer is a simple number of the highest dilution factor that produced a positive reading. Meaning that the highest dilution factor would be the well before the dot/button.

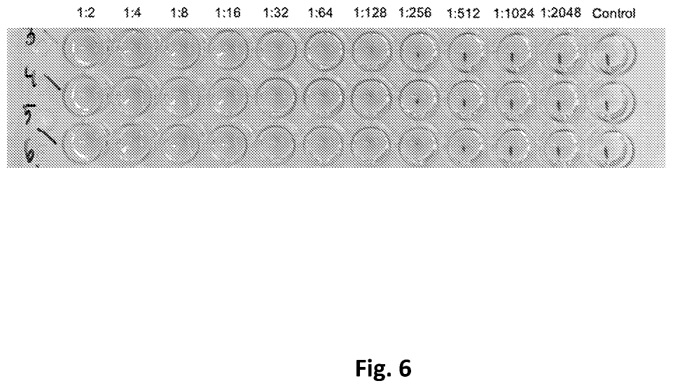

FIG. 6 illustrates a typical HA assay. Referring to FIG. 2: Lane 3 and 4: Spray dried material showing a titer of 128 (1:128), this corresponds to the sample after Spray drying. Lane 5—lyophilized material in PBS, showing a titer of at least 128 (1:128), potentially 256 (1:256).

For Example 2, the EDS virus Strain VLDIA038 HAG EDS'76, Adeno 127 HI antigen, Lot no. 19658-260319 with Exp. date 03-2029 from GD Animal Health was used. The lyophilized EDS virus was reconstituted and diluted with buffer 1—containing 10 mM Tris, 75 mM NaCl, 0.1 mM EDTA, 10 mM Histidine, 1 mM MgCl₂ and 0.02% Tween 80 and then upconcentrated 20× from 12 ml to 600 μl using centrifugal concentrator vials with a cut-off of 100,000 MWCO, in order to remove smaller particles. For spray drying, the upconcentrated sample was further diluted in order to have material enough to spray dry; 500 μl was mixed with 4.5 ml buffer 1+ excipient to a final concentration of 2% 2-hydroxypropyl-beta-cyclodextrin (HPBCD). The spray dried sample was therefore 10x diluted compared to the upconcentrated sample. The remaining material was used for comparison of virus that has not been processed with the drying process and the virus material yielded by the spray drying process.

The sample for spray drying was applied to the nebulizer of the Laminar Pace instrument and spray dried (see http://www.inhalation.se/produkter/laminarpace/). The material was generated in two drying processes, drying 3 ml and 2 ml. The settings of the instrument is demonstrated in Table 1 below.

The dried material was collected on a pre-weighted nylon filter at the bottom of the Laminar Pace column. At the end of the drying process the formed powder was weighed. The yield was determined from the theoretical amount of buffer/excipient (27.6 mg/ml)

TABLE 1

| Process | Parameter | Run 1 | Run 2 |
|---|---|---|---|
| At preparation | Temp/° C. | 23.7 | 23.3 |
| | Relative humidity/% | 26.0 | 26.8 |
| Running LaPa | Production rate/% | 37 | 37 |
| | Nebulizer flow/(L/min) | 2.11 | 2,10 |
| | Nebulizer vacuum/(mm | 0 | 0 |
| | v.p.) | | |
| | Initial filter dP/mbar | 49.03 | 54.25 |
| | After filter dP/mbar | 55.14 | 58.97 |
| | Initial Hum1/% | 1.73 | 3.92 |
| | After Hum1/% | 4.92 | 4.85 |
| | Initial Hum2/% | 2.12 | 3.75 |
| | After Hum 2/% | 3.34 | 3.93 |
| Results | Filter paper/mg | 121.7 | 132.7 |
| | Filter paper + | 196.0 | 179.1 |
| | excipient/mg | | |
| | Excipient + API/mg | 74.3 | 46.4 |
| | Theoretical | 82.8 | 55.2 |
| | Excipient + API/mg | | |
| | Added volume/ml | 3 | 2 |
| | Yield/% | 89.7 | 84.1 |
| At collection | Temp/° C. | 23.3 | 23.2 |
| | Relative humidity/% | 26.9 | 26.4 |

Samples of spray dried material were stored at 4° C. and 40° C. The samples were tested with HA analysis performing the Viral dilution and assay, as described above on Day 0, 3, 7, 23 and 51. The HA analysis on day 0 also included a comparison in virus activity between material before and after spray drying. In Table 2, below, the resulting titers of the HA analysis demonstrate that both spray dried and non spray dried material have the same titer. In other terms, the spray drying method used with the invention does not reduce the virus activity for the differently diluted samples compared to non-treated material. Further Table 2 demonstrates that the titer is the same over 3 to 51 days at both at 4° C. and 40° C. which means that virus activity is retained also at higher temperatures, indicating that compositions in accordance with the invention are capable of being thermostable.

TABLE 2

| | Day 0 | | | | | | | | | | | |
|---|---|---|---|---|---|---|---|---|---|---|---|---|
| | Before | | | | | | | | | | | |
| | spray | | Day 0 | | Day 3 | | Day 7 | | Day 23 | | Day 51 | |
| | drying | After | Before | After | 4° | 40° | 4° | 40° | 4° | 40° | 4° | 40° |
| Dilution | (SD) | SD | SD | SD | C. | C. | C. | C. | C. | C. | C. | C. |
| 1 | 256 | 256 | 512 | 512 | 512 | 512 | 512 | 512 | 512 | 512 | 512 | 512 |
| 2 | 256 | 256 | 256- | Not | 512 | 512 | 512 | 512 | 512 | 512 | 512 | 512 |
| | | | 512 | analyzed | | | | | | | | |

In Table 3, below the thermostability of the lyophilized composition of the EDS virus strain (GD animal health) was stored at 4° C. and 40° C. HA analysis as outlined above was performed after 3 and 7 days. The results of Table 3 indicate that the titer is dramatically reduced after 7 days at 40° C. and that the lyophilized compositions is not capable of inducing thermostability of the virus. In comparison, the compositions according to the present invention as demonstrated in Table 2 are capable of maintaining virus activity throughout storage at 40° C. In conclusion, the results of Example 2 demonstrated in Tables 2 and 3 confirm that the invention as described and claimed provides highly stable virus compositions useful as vaccines and in other therapeutic applications.

TABLE 3

| | Day 3 | | Day 7 | |
|---|---|---|---|---|
| Dilution | 4° C. | 40° C. | 4° C. | 40° C. |
| 2 | 1024 | 128 | 2048 | 16 |
| 10 | 1280 | 80 | 1280 | 20 |

### Example 3

Preliminary experiments were made with an extract of Inactivated EDS Adenovirus: From Hester Biosciences Limited, Gujarat, India (see Example 1) and albumin, a dextran and glycine, respectively, as the excipient in similar or the same concentrations as HPBCD in the previous Examples and with the same spray drying process as defined in Example 1 and 2. The results demonstrated a high yield following the spray drying process and an immediately maintained activity of the virus with the HA assay. However, in contrast to Examples 1 and 2 where HPBC was used as an excipient, virus activity was significantly lost during storage at 40° C. These results confirm that the above described Laminar Pace process for spray drying admits favorable drying conditions for maintaining virus activity and yield, while the desired thermostability at 40° C. was not admitted by any of these excipients under the given circumstances.

1. A thermostable dry composition comprising aerosolizable amorphous particles, comprising free, non-encapsulated noneveloped virus and an excipient, wherein the particles:
have a mass median aerodynamic diameter (MMAD) of less than 5 μm;
comprise less than 5% (wt) water; and wherein
the nonenveloped virus of the dry particles maintain its activity following storage at 40° C. for at least seven weeks.

2. The composition according to claim 1, wherein the excipient comprises a cyclodextrin.

3. The composition according to claim 2, comprising at least 90% (wt) of the excipient.

4. The composition according to claim 2, wherein the excipient comprises 2-hydroxypropyl-beta-cyclodextrin.

5. The composition according to claim 1, wherein the excipient is free from amino acids

6. The composition according to claim 1, wherein the excipient is free from mono- and disaccharides.

7. The composition according to claim 5, free from glycine and mannitol.

8. The composition according to claim 1, wherein the nonenveloped virus is an adenovirus.

9. The composition according to claim 1, adapted to pulmonary administration in an aerosolizable form.

10. A method of preparing a thermostable aerosolizable dry nonenveloped virus composition by a counter-current spray drying process, the method comprising:
providing a liquid composition comprising nonenveloped virus and an excipient;
nebulizing the composition into transportable droplets of less than 50 μm in a tube reactor having an inner region and an outer region;
admitting the nebulized composition to descend in a laminar carrier gas flow while admitting a laminar flow of dry gas to ascend in the outer region in order to establish a counter-current drying of the descending drops;
drying the droplets for 30 seconds to 2 minutes at an ambient temperature from 15° C. to 30° C. while admitting vapour to diffuse into the ascending laminar flow, thereby producing a dry composition of particles and
collecting the dry composition of particles.

11. The method according to claim 10, wherein the inner region and the outer region tube reactor are separated by perforated process tube with an outer periphery covered by a membrane configured to admit vapour to diffuse through the membrane into the ascending laminar flow of dry air at a rate that exceeds an opposite flow rate of dry gas radially through the membrane.

12. The method according to claim 10, wherein the liquid composition comprises 0.5 to 5% (wt) of a cyclodextrin.

13. The method according to claim 10, wherein the ascending flow rate is higher than the descending flow rate.

14. The method according to any one of claim 10, wherein the ascending flow performs swirls around the membrane.

15. The method according to claim 10, wherein providing the liquid composition comprises:
preparing the liquid virus composition by:
removing hydrophobic agents that contribute to encapsulation or aggregation of enveloped virus; and
adding a suitable excipient.

16. The method according to claim 15, further comprising adding a surfactant and removing micelles with the hydrophobic agents.

17. The method according to claim 10, wherein the yield of the spray drying process is at least 80% as a calculated from a theoretical amount of the provided liquid virus composition.

18. (canceled)

19. The composition according to claim 1, wherein the particles have a mass median aerodynamic diameter (MMAD) of from about 1 μm to 5 μm.

20. The composition according to claim 19, wherein the particles have a mass median aerodynamic diameter (MMAD) of from 2 μm to 3 μm.

21. The method according to claim 12, wherein the cyclodextrin comprises 2-hydroxypropyl-beta-cyclodextrin.
